(19) United States
(12) Plant Patent Application Publication
(10) Pub. No.: US 2012/0246775 P1
(43) Pub. Date: Sep. 27, 2012

(54) Chrysanthemum named 'Plum Crazy'

(50) ;

(75) Inventor: Celine Emmanuelle Veronique Pestail, Chichester (GB)

(73) Assignee: Chrysanthemum Breeders Association Research B.V., ZG Valkenburg Z-H (NL)

(21) Appl. No.: 13/064,408

(22) Filed: Mar. 23, 2011

(51) Int. Cl. A01H 5/00

(52) U.S. Cl. PLT/287

(57) ABSTRACT

A new variety of Chrysanthemum plant named ‘Plum Crazy’, characterized by its good uniform canopy of dark purple flowers. The new variety has a medium vigor, a free branching and uniform spreading habit, a good foliage presentation and flowers week 38 in natural season.

### LATIN NAME OF THE GENUS AND SPECIES

### Botanical Classification

Chrysanthemum×morifolium Ramat.

### VARIETY DENOMINATION

The new Chrysanthemum variety denomination is ‘Plum Crazy’.

### BACKGROUND OF THE INVENTION

The present invention comprises a new and distinct cultivar of Chrysanthemum botanically known as Chrysanthemum×morifolium Ramat., and referred to by the cultivar name ‘Plum Crazy’, identified as 30769-0 originated from a cross made in a controlled breeding program in Chichester, West Sussex, United Kingdom. The new and cultivar was discovered and selected as a flowering plant in September 2000.

The female parent of the new variety is an undistributed, unpatented seedling identified as G69D2 and the male parent is the unpatented variety ‘Cyblis’.

The new variety ‘Plum Crazy’ has been asexually reproduced by vegetative cuttings in Chichester, West Sussex, United Kingdom and the distinguishing characteristics are retained through successive generations of asexual reproduction.

### BRIEF SUMMARY OF THE INVENTION

‘Plum Crazy’ is a garden type of Chrysanthemum plant variety having a uniformed domed capitulum of dark purple decorative type flowers.

### Comparison with Female Parent

Plants of the new Chrysanthemum variety ‘Plum Crazy’ are dissimilar to the female parent ‘G69D2’ in plant habit and growth rate. In side-by-side comparisons in Chichester, West Sussex, United Kingdom, under commercial practice, plants of the new Chrysanthemum variety ‘Plum Crazy’ differed from plants of the female parent ‘G69D2’ in the following characteristics:
1. The new variety ‘Plum Crazy’ produces purple decorative flowers whereas the female parent ‘G69D2’ produces pink decorative flowers.
2. In natural growth condition, plants of the new variety ‘Plum Crazy’ show flowers one week after plants of the female parent ‘G69D2’.
3. The plants of the new variety ‘Plum Crazy’ have a more uniform growing habit than the plants of the female parent ‘G69D2’.

### Comparison with Male Parent

Plants of the new Chrysanthemum variety ‘Plum Crazy’ are similar to the male parent ‘Cyblis’ in plant habit and growth rate. However, in side-by-side comparisons in Chichester, West Sussex, United Kingdom, under commercial practice, plants of the new Chrysanthemum variety ‘Plum Crazy’ differed from plants of the male parent ‘Cyblis’ in the following characteristics:
1. The new variety ‘Plum Crazy’ produces purple decorative flowers whereas the male parent ‘Cyblis’ produces purple daisy flowers.
2. In natural growth condition, plants of the new variety ‘Plum Crazy” show flowers one week before plants of the male parent ‘Cyblis’.

### Comparison with Most Similar Variety

Plants of the new Chrysanthemum variety ‘Plum Crazy’ are similar to plants of the variety ‘Jenny Wren Crimson’ U.S. Plant Pat. No. 16,035 in plant habit and growth rate. However, in side-by-side comparisons in Chichester, West Sussex, United Kingdom, under commercial practice, plants of the new Chrysanthemum variety ‘Plum Crazy’ differed from plants of ‘Jenny Wren Crimson’ in the following characteristics:
1. The new variety ‘Plum Crazy’ produces purple flowers and the variety ‘Jenny Wren Crimson’ produces crimson flowers.
2. Plants of the new variety ‘Plum Crazy’ have larger blooms than the plants of the ‘Jenny Wren Crimson’.
3. In natural growth condition, plants of the new variety ‘Plum Crazy’ flower three weeks later than plants of ‘Jenny Wren Crimson’.

### BRIEF DESCRIPTION OF ILLUSTRATIONS

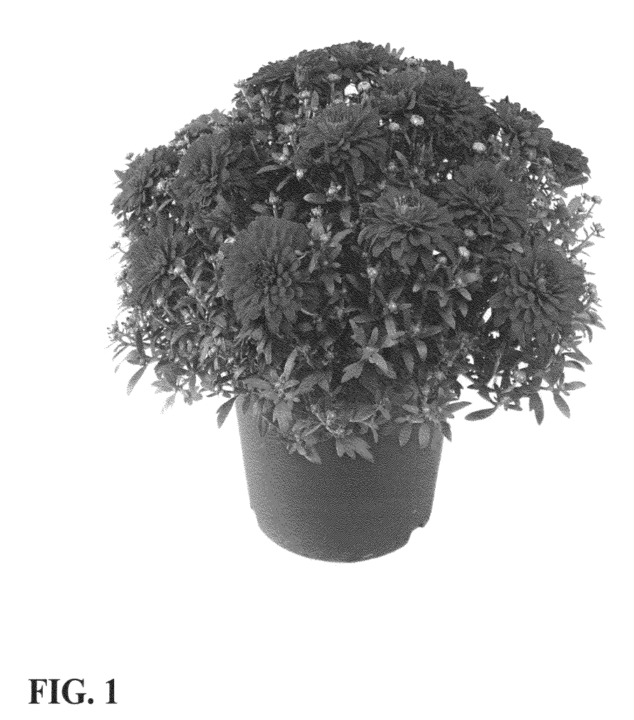

Typical specimens of the plant and flowers for the new Chrysanthemum variety ‘Plum Crazy’ are shown in the accompanying photograph. The colors shown are as true as possible within the usual limits of this kind of illustration. FIG. 1 is a whole plant view of the new variety ‘Plum Crazy’ grown in a pot. The plant shown in the illustration is around 90 days from the commencement of planting outdoors in natural season conditions.

### DETAILED BOTANICAL DESCRIPTION

The following description of the new Chrysanthemum variety ‘Plum Crazy’ is of plants grown outside, natural season, in Chichester, West Sussex, United Kingdom in the month of September in 2010. The cultivar has not been observed under all possible environmental conditions. The phenotype may vary significantly with variations in the environment such as temperature, length of day and light intensity, without any variance in genotype. The commercial classification of the new variety is a garden Chrysanthemum.

Plants of the new variety have been grown successfully under temperature conditions averaging about 10° C. at night and about 10° C. to 25° C. during the day. The plants were grown outdoors in 4 liter pots using a loamless compost. No growth retardants were used.

To produce a commercial product, the plants need not to be pinched. The typical container size for commercial growth is 4 liters. It has been observed that the shelf life of the new variety is about 28 days with a response time of about 13 weeks natural season. The new variety is suitable for a temperature range of 10° C. to 25° C.

The new variety may be produced as a garden pot plant. The following description is with respect to a plant produced as a garden pot plant. In the description of this new Chrysanthemum variety, color values have been taken from The Royal Horticultural Society Colour Chart, edition 2001.
Plant:
   Plant type.—Pot.
   Habit.—Domed, free branching and spreading.
   Height (cm).—25.
   Width (cm).—45.
   Stem.—Length (cm): 12 Diameter (cm): 0.5-1 Texture: Rough Color: near Grey-brown 199B.
   Branching characteristics.—Free branching and spreading.
   Lateral branches.—Length (cm): 6-13 Diameter (cm): 0.5-0.7 Texture: Slightly rough.
   Natural season flowering date.—week 38.
   Vigor.—medium to tall.
   Shelf life.—28 days.
   Life of blooms.—28 days.
   Disease (susceptibility/resistance observed).—None observed to date.
   Pest (susceptibility/resistance observed).—None observed to date.
   Drought and temperature (susceptibility/resistance observed).—None observed to date.
   Growth retardant type and treatment.—No treatment necessary The plants were grown for 14 weeks in natural season conditions.
Propagation:
   Type.—Vegetative propagation via stem cuttings.
   Time to rooting.—12 days with soil temperatures of 21° C.
   Rooting habit.—In 12 long days, a complete root system is developed.
Foliage:
   Number of leaves per lateral branch.—6-9.
   Compound or single.—Single.
   Arrangement of leaves.—Alternate Shape of Leaf: Typically 5 lobed ovate Size of Leaf: Width (cm): 1.8-3.8; Length (cm): 3-4.5 Leaf Apex: Acute Base: Corbate Attachment: Petioled Aspect: Slightly undulating Margin: Palmately lobed Surface characteristics: Top: Slightly pubescent Bottom: Slightly pubescent.
   Petiole.—Color: near Yellow-green 148A Length (cm): 0.8-1 Diameter (cm): 0.1-0.2 Surface: Slightly pubescent.
   Venation.—Net, prominent mid-veins at underside Color: upper side: near Yellow-green148A under side: near Yellow-green148A.
   Color.—Mature Leaf, upper side: near Yellow-green 147A; under side: near Yellow-green 147B Young Leaf, upper side: near Yellow-green 147A; under side: near Yellow-green 147B.
Inflorescence:
   Inflorescence appearance.—Matte.
   Inflorescence type.—Single.
   Inflorescence form.—Slightly cupped.
   Inflorescence shape.—Round.
   Flowering habit.—Cyme.
   Number of inflorescences per branch.—6-15.
   Depth of fully expanded inflorescences (cm).—1.
   Diameter of fully expanded inflorescences (cm).—7.
   Phyllaries.—Number: 20 Color: near Yellow-green147A Length (cm): 0.4-0.6 Width (cm): 0.1-0.2 Texture/Appearance: Shiny/Slightly pubescent.
   Pedicel.—Length: terminal (cm): 4-6; lateral (cm): 1-3 Diameter (cm): 0.2 Angle from stem (degrees): 45 Color: near Yellow-green 146B Surface: Slightly pubescent Habit: Slightly curving Strength: Medium.
   Ray florets.—Form/Shape: Straight Texture/Appearance: Matte Number per flower: about 700 Length (cm): 2-3; Width (cm): 1-3 Apex: Rounded Base: Tubular Margin: Entire.
   Disc florets.—Form/Shape: Cylindrical Texture/Appearance: Matte Number per flower: 8-15 Length (cm): 0.3; Width (cm): 0.1 Diameter of disc (cm): 0.3.
   Fragrance.—None.
   Flower bud (at onset of color).—Length (cm): 0.7 Diameter (cm): 0.9 Form/Shape: Domed flat.
   General flower color.—1. Ray florets, upper side: Immature: near Red-purple 59B Mature: near Red-purple 61A Older/Fading: near Red-purple 71A 2. Ray florets, under side: Immature: near Purple 77B Mature: near Purple 77B Older/Fading: near Purple 77C 3. Bud: near 77B.
   Flower progression with age.—When the flower ages, the ray florets are reflexing and becoming lighter.
Reproductive organs: Gynoecium present on Ray florets
   Ray florets per individual flower.—Pistillate Pistil number: near 700 Stigma color: near Yellow 9A Stigma shape: Forked Style color: near Yellow 9B Style length (cm): 0.3.
   Disc florets per individual flower.—Pistillate Pistil number: 8-15 Stigma color: near Yellow-orange 14A Stigma shape: Forked Style color: near Yellow-green 154B Style length (cm): 0.4.
   Androecium present on disc florets only.—Staminate Stamen number: 8-15 Anther shape: Clubbed Anther color: Near Yellow-orange 17B Pollen color: Near Yellow-orange 14A Pollen quantity: None.
   Seeds.—None observed.

What is claimed is:

1. A new and distinct variety of Chrysanthemum plant, substantially as described and illustrated herein.
